(19) United States
(12) Patent Application Publication
(10) Pub. No.: US 2016/0044914 A1
(43) Pub. Date: Feb. 18, 2016

(54) BIOCIDE COMPOSITIONS, METHODS OF MANUFACTURE, AND METHODS OF USE

(71) Applicant: SEGETIS, INC., Golden Valley, MN (US)

(72) Inventors: DORIE J. YONTZ, BLOOMINGTON, MN (US); TIMOTHY M. CUSACK, WEST LAKELAND, MN (US); ATUL C. THAKRAR, THE HAGUE (NL)

(21) Appl. No.: 14/776,803

(22) PCT Filed: Mar. 14, 2014
(86) PCT No.: PCT/US2014/027117

§ 371 Date: Sep. 15, 2015

Related U.S. Application Data
(60) Provisional application No. 61/794,126, filed on Mar. 15, 2013.
(60) Provisional application No. 61/789,751, filed on Mar. 15, 2013.

(51) Int. Cl. A01N 25/02; C07D 317/30; C07C 69/716; C07D 275/02; C07D 307/06; A01N 57/20; A01N 39/04; A01N 37/40; A01N 41/10; A01N 47/38; A01N 41/06; A01N 57/12; A01N 53/00; A01N 43/653; A01N 37/22; A01N 25/04; C07D 317/20

(52) U.S. Cl. CPC A01N 25/02; C07D 317/20; C07D 317/30; C07C 69/716; C07D 275/02; C07D 307/06; A01N 57/20; A01N 39/04; A01N 37/40; A01N 41/10; A01N 47/38; A01N 41/06; A01N 57/12; A01N 53/00; A01N 43/653; A01N 37/22; A01N 25/04

(57) ABSTRACT

A biocide composition comprises a biocide and an adjuvant comprising a ketal having the formula (1)

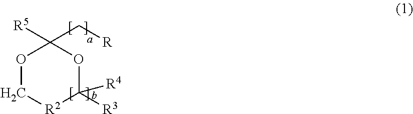

wherein a is 0 or an integer of 1 to 12, specifically 1 to 6, more specifically 1 to 4, still more specifically 2 to 4; b is 0, 1, or 2; R is —C(═O)OR¹ or —CH₂OH; R¹ is a C₁₋₁₈ alkyl or C₅₋₈ cycloaliphatic or heterocyclic group; R² is a divalent C₁₋₈ alkylene group optionally substituted with up to 5 hydroxyl groups; R³ and R⁴ are each independently hydrogen or C₁₋₆ alkyl; and R⁵ is hydrogen or C₁₋₃ alkyl.

### PRIORITY

This application is a National Stage application of PCT/US2014/027117, filed Mar. 14, 2014, which claims priority to U.S. Provisional Patent Application Ser. No. 61/789,751, filed on Mar. 15, 2013 and to U.S. Provisional Patent Application Ser. No. 61/794,126, filed on Mar. 15, 2013, all of which are hereby incorporated by reference in their entirety for all purposes.

### BACKGROUND OF THE INVENTION

This disclosure relates to biocide compositions containing a biocide and a formulation enhancer (adjuvant) comprising a ketal having ester and/or hydroxyl functionality, or a heterocyclic compound.

Biocides, including insecticides, herbicides, and fungicides, are important materials in agriculture. They are used to protect crops from pests and thereby increase crop yield. Biocides are frequently formulated with inactive components, such as diluents and adjuvants (also commonly referred to as co-formulants). While not being used as the primary biocidal active itself, an adjuvant can increase the efficacy of a biocide is mixed with. Biocides are often supplied as concentrates, which are designed to be diluted with water to the desired concentration of biocide by the end-user. Water is frequently used as a diluent for biocide concentrates. The use of water is desirable because water is relatively inexpensive, environmentally safe, and compatible with further dilution of the concentrate with water by the end-user before spraying. Biocide compositions comprising diluent and adjuvants can be applied by the end-user to the target crops or turf, undiluted or after dilution with water. Liquid biocide concentrates, for example emulsifiable concentrates, suspension concentrates, and suspo-emulsion concentrates (combination of dispersed solid particles and emulsified liquid particles) are often used because they are readily measured, poured, and diluted with water. When diluted with water, biocide concentrates form readily sprayable aqueous solutions, oil-in-water emulsions, aqueous suspensions, or suspo-emulsions.

A major drawback of biocide concentrates is storage stability. Solid biocides can crystallize from the oil phase of emulsifiable concentrates and settle out. The solid biocide particles of suspension concentrates and suspo-emulsion concentrates can agglomerate and flocculate over time, leading to settling of the solid particles in the concentrate containers. The settling of the solid particles can lead to the creation of hard-packed sediment at the bottom of concentrate containers making it difficult to remove the biocide from the container. Even if the solid biocide particles remain suspended in the concentrate, the particles can aggregate and settle out upon dilution with water by the end-user. The rate of sedimentation in the end-user's storage tank depends on a number of factors including particle size, particle concentration, viscosity of the continuous phase, and the specific gravity difference between the particles and the continuous phase. Once settled in the end-user's tank, the sediment can become hard-packed, making redispersion extremely difficult. The creation of hard-packed sediment can occur when the tanks are not agitated during interruptions in sprayings. Even if the sediment does not become hard-packed the agglomerated solids particles can plug filters and spray nozzles present in the spray equipment.

Moreover, there is an increasing demand for adjuvants derived from renewable resources such as biomass, often described as “bio-sourced”, that can be used as replacements for petroleum-sourced adjuvants. Few bio-sourced adjuvants are available that can meet the increasingly demanding technical requirements for biocide compositions. Even when bio-sourced adjuvants are available, these adjuvants can have various drawbacks. For example, ethanol is a versatile solvent that is readily available from biomass such as corn, but its low flash point and high flammability limits its use biocide compositions. A further drawback of existing bio-sourced adjuvants is that the there is limited ability to chemically modify the adjuvants to obtain improved physical properties. Runoff of toxic solvents in biocide compositions to steams, rivers, ponds, and lakes can present a pollution problem. Therefore there is also an increasing demand for adjuvants that are non-toxic.

While a number of adjuvants useful for biocide compositions are commercially available, there remains a need in the art for an adjuvant which can stabilize biocide compositions against crystallization or agglomeration and settling of solid biocide particles. It would be a further advantage for the adjuvant to be bio-sourced, to have good solvency for a wide variety of biocides, to have increased resistance to water wash-off after application of the biocide composition, and increased penetration of the biocide into plant leaves. It is also desirable for the adjuvant to be chemically inert under normal conditions of manufacture, storage, and use, and to be unreactive with other components commonly found in biocide compositions. It is also desirable for the adjuvant to be nontoxic, biodegradable, and to have low volatility and low cost. It would also be advantageous if it were easy to incorporate a wide variety of substituents into the adjuvant chemical structure so that the above physical properties could be optimized for each application.

### SUMMARY OF THE INVENTION

Disclosed herein is a biocide composition comprising a biocide, and an adjuvant comprising a ketal having the formula (1)

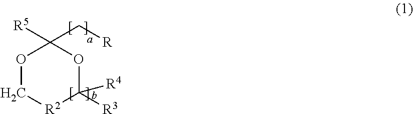

wherein a is 0 or an integer of 1 to 12, specifically 1 to 6, more specifically 1 to 4, still more specifically 2 to 4; b is 0, 1, or 2; R is —C(═O)OR¹ or —CH₂OH; R¹ is a C₁₋₁₈ alkyl or C₅₋₈ cycloaliphatic or heterocyclic group; R² is a divalent C₁₋₈ alkylene group optionally substituted with up to 5 hydroxyl groups; R³ and R⁴ are each independently hydrogen or C₁₋₆ alkyl; and R⁵ is hydrogen or C₁₋₃ alkyl; a compound having the formula (6)

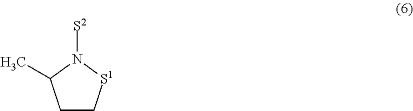

wherein S¹ is >C═O (carbonyl) or >CH₂ (methylene), and S² is C₁₋₁₂ alkyl, C₆₋₂₀ cycloalkyl, or C₆₋₂₀ aryl; a compound having the formula (7) or (8)

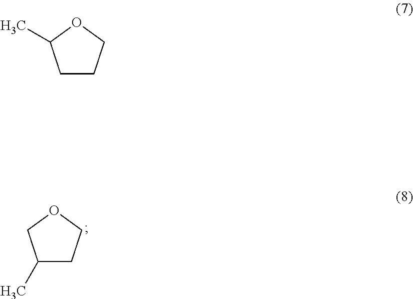

or a combination thereof.

Also disclosed herein is a biocide composition comprising:

a biocide, and

an adjuvant comprising a compound having the formula (11)

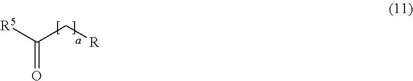

wherein

a is 0 or an integer of 1 to 12, specifically 1 to 6, more specifically 1 to 4, still more specifically 2 to 4, still more specifically 2;

R is —CH₂OH; and

R⁵ is hydrogen or C₁₋₃ alkyl.

Also disclosed herein is a biocide composition comprising:

a biocide, and

an adjuvant comprising a compound having the formula (3)

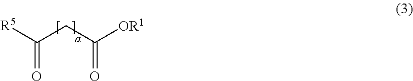

wherein

a is 0 or an integer of 1 to 12, specifically 1 to 6, more specifically 1 to 4, still more specifically 2 to 4, still more specifically 2;

R¹ is a C₁₋₁₈ alkyl or C₅₋₈ cycloaliphatic or heterocyclic group; and

R⁵ is hydrogen or C₁₋₃ alkyl.

Also disclosed herein is an emulsifiable concentrate comprising the biocide, the adjuvant, a surfactant; and optionally an oil.

Also disclosed herein is a suspension concentrate comprising the biocide, the adjuvant, water, a surfactant; and optionally an oil.

Also disclosed herein is a tank mix composition for foliar or soil application, comprising the biocide, the adjuvant, water, a surfactant, and optionally an oil.

Also disclosed herein is a ready-to-use liquid composition comprising a solvent other than the adjuvant; and optionally a thickening agent, a propellant, an attractant, or a combination thereof, wherein the solvent is 90% vaporized within 5 minutes of application of the composition to a surface.

Also disclosed herein is a crop oil concentrate, comprising the adjuvant.

Also disclosed herein is a seed treatment composition, comprising the adjuvant.

A method of forming a tank mix composition comprising mixing the crop oil concentrate with an emulsifiable concentrate, a suspension concentrate, a suspo-emulsion concentrate, a wettable powder, a dry flowable powder, or a soluble powder, and water to a desired end-use concentration of biocide is also disclosed.

Also disclosed herein is a seed treatment composition comprising the biocide, and the adjuvant.

Also disclosed herein is a method of controlling a pest comprising contacting the pest or the locus of the pest with the biocide composition, in an amount effective to control the pest.

The invention is also directed to biocide formulations in the form of any of the following: aqueous solutions, emulsifiable concentrates, emulsifiable granules, suspension concentrates, water dispersible granules, wettable powders, granules, oil in water emulsions, suspension emulsions, microemulsions, oil dispersions, and capsule suspensions.

The above described and other embodiments are further described by the drawings, detailed description, and claims.

### DETAILED DESCRIPTION OF THE INVENTION

The present inventors have discovered new adjuvants for use in biocide compositions. These adjuvants provide several improvements in the properties of biocide compositions. The adjuvants can stabilize biocide compositions against crystallization or agglomeration and settling of solid biocide particles. The adjuvants are also bio-sourced, have good solvency for a wide variety of biocides, and can increase penetration of the biocide into plant leaves. The adjuvants are chemically inert under normal conditions of manufacture, storage, and use, and are unreactive with other components often present in biocide compositions. The adjuvants are biodegradable and derived from renewable resources such as biomass. The adjuvants also have low toxicity, volatility, and cost. Moreover, the chemical structure of the adjuvants makes it easy to incorporate a wide variety of different substituents into the adjuvant chemical structure so that the above physical properties can be optimized for any particular application.

Thus, a biocide composition comprises a biocide and an adjuvant comprising a ketal having the general formula (1)

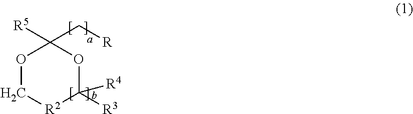

wherein a is 0 or an integer of 1 to 12, specifically 1 to 6, more specifically 1 to 4, still more specifically 2 to 4; b is 0, 1, or 2; R is —C(═O)OR¹ or —CH₂OH; R¹ is a C₁₋₁₈ alkyl or C₅₋₈ cycloaliphatic or heterocyclic group; R² is a divalent C₁₋₈ alkylene group optionally substituted with up to 5 hydroxyl groups; R³ and R⁴ are each independently hydrogen or C₁₋₆ alkyl; and R⁵ is hydrogen or C₁₋₃ alkyl.

In some embodiments the adjuvant is a “ketal ester”, wherein R is —C(═O)OR¹ and R¹ is defined as above. In some embodiments the adjuvant is a “ketal alcohol”, wherein R is —CH₂OH. R² can be >CH—CH₃, >CH—CH₂OH, >C(CH₃)CH₂OH, >C(C₂H₅)CH₂OH, >C(CH₂OH)₂, >CH—CH(OH)—CH₂OH, or >CH—(CHOH)₃—CH₂OH.

Specifically, R² is —C(R⁶)(R⁷)—, wherein R⁶ is hydrogen, C₁₋₃ alkyl, or —CH₂OH, and R⁷ is hydrogen; R³ and R⁴ are each independently hydrogen or C₁₋₃ alkyl; R⁵ is methyl; a is 1, 2, 3, or 4; and b is 0 or 1.

More specifically, R² is —C(R⁶)(R⁷) wherein R⁶ is hydrogen, C₁₋₃ alkyl, or —CH₂OH, and R⁷ is hydrogen; R⁵ is methyl; a is 2 or 3; and b is 0.

Even more specifically, R² is —C(R⁶)(R⁷) wherein R⁶ is methyl, ethyl, or —CH₂OH, and R⁷ is hydrogen; R⁵ is methyl; a is 2; and b is 0.

In specific embodiments, the ketal is a ketal ester having the formula (1a)

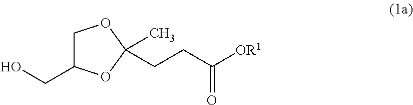

or the formula (1b)

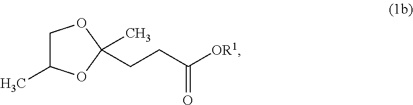

wherein R¹ is methyl, ethyl, n-propyl, n-butyl, 2-ethylhexyl, 1-nonyl, 3,5,5-trimethylhexyl, or tetrahydrofurfuryl.

In other embodiments, the ketal is a ketal alcohol having the formula (1c)

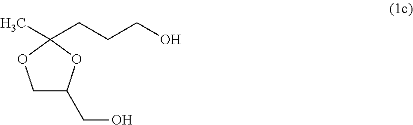

or the formula (1d)

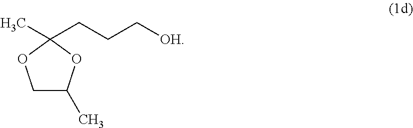

The adjuvant can be the ketal ester of formula (1a) or formula (1b), the ketal alcohol of formula (1c) or formula (1d), or a combination thereof.

The adjuvant can be the ketal ester of formula (1a) or formula (1b), the ketal alcohol of formula (1c) or formula (1d), or a combination thereof.

In other embodiments, adjuvant can comprise a compound having the formula (11)

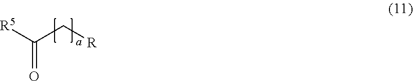

wherein

a is 0 or an integer of 1 to 12, specifically 1 to 6, more specifically 1 to 4, still more specifically 2 to 4, still more specifically 2;

R is —CH₂OH; and

R⁵ is hydrogen or C₁₋₃ alkyl.

Specifically, R⁵ is methyl; and a is 1, 2, 3, or 4. More specifically, R⁵ is methyl; and a is 2 or 3. Even more specifically, R⁵ is methyl; and a is 2.

In some embodiments, the adjuvant is a ketoalcohol having the formula (11a)

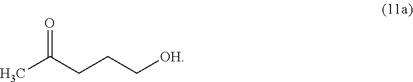

In other embodiments, adjuvant can comprise a compound having the formula (3)

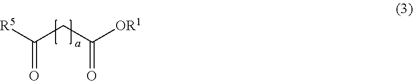

wherein

a is 0 or an integer of 1 to 12, specifically 1 to 6, more specifically 1 to 4, still more specifically 2 to 4, still more specifically 2;

R¹ is a C₁₋₁₈ alkyl or C₅₋₈ cycloaliphatic or heterocyclic group; and

R⁵ is hydrogen or C₁₋₃ alkyl.

Specifically, R⁵ is methyl; and a is 1, 2, 3, or 4. More specifically, R⁵ is methyl; and a is 2 or 3. Even more specifically, R⁵ is methyl; and a is 2.

Specifically, R¹ is a C₁₋₄ alkyl, more specifically, a C₂ alkyl. More specifically, R¹ is a C₁₋₄ alkyl, R⁵ methyl; and a is 1, 2, 3, or 4. More specifically, R¹ is a C₁₋₄ alkyl, R⁵ is methyl; and a is 2 or 3. Even more specifically, R¹ is a C₁₋₄ alkyl, R⁵ is methyl; and a is 2. Even more specifically, R¹ is a C₂ alkyl, R⁵ is methyl; and a is 2. In other embodiments, R¹ is a C₅₋₁₈ alkyl, more specifically, a C₅₋₁₂ alkyl and more specifically, a C₈₋₁₂ alkyl. More specifically, R¹ is a C₅₋₁₂ alkyl, R⁵ methyl; and a is 1, 2, 3, or 4. More specifically, R¹ is a C₅₋₁₂ alkyl, R⁵ is methyl; and a is 2 or 3. Even more specifically, R¹ is a C₅₋₁₂ alkyl, R⁵ is methyl; and a is 2. Even more specifically, R¹ is a C₈₋₁₂, R⁵ is methyl; and a is 2.

In some embodiments, the adjuvant has the formula (3a)

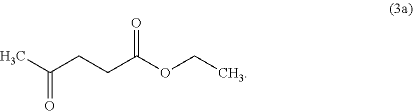

In some embodiments the adjuvant is a compound having the formula (6)

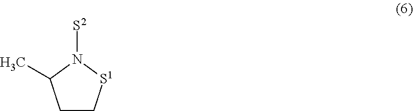

wherein S¹ is >C═O (carbonyl) or >CH₂ (methylene), and S² is C₁₋₁₂ alkyl, C₆₋₂₀ cycloalkyl, or C₆₋₂₀ aryl; a compound having the formula (7) or (8)

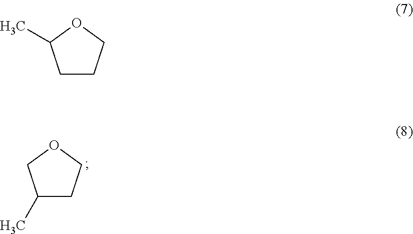

or a combination thereof.

In some embodiments, the adjuvant comprises the compound having the formula (6), wherein S² is methyl, ethyl, butyl, octyl, decyl, dodecyl, phenyl, 4-methylphenyl, or cyclohexyl; or wherein S¹ is >C═O (carbonyl) and S² is methyl, ethyl, butyl, octyl, decyl, dodecyl, phenyl, 4-methylphenyl, or cyclohexyl; or wherein S¹ is >CH₂ (methylene) and S² is methyl, ethyl, butyl, octyl, decyl, dodecyl, phenyl, 4-methylphenyl, or cyclohexyl.

Specifically, the adjuvant can comprise the compound of formula (9)

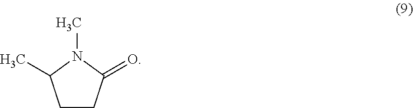

The adjuvant can also comprise the compound of formula (10)

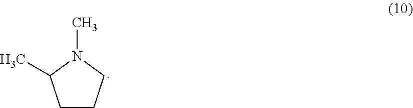

The adjuvant can also comprise the compound of formula (7)

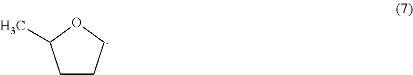

The adjuvant can also comprise the compound of formula (8)

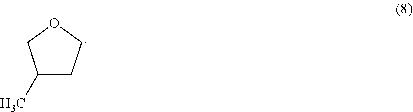

The ketal alcohol of formula (1) wherein R is —CH₂OH can be obtained by the reduction of the corresponding ketal ester (2)

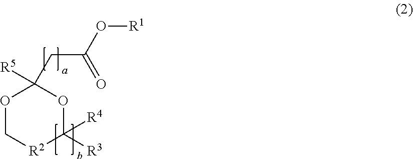

wherein R¹, R², R³, R⁴, R⁵, and a and b are as defined for formula (1). Reduction can be carried out, for example, by a hydride such as LiBH₄ or LiAlH₄. Alternatively, the ketal alcohol can be obtained by reducing ketal ester (2) under catalytic hydrogenation conditions. Exemplary catalysts for the hydrogenation include homogeneous ruthenium catalysts, and copper chromite catalyst. Ketal ester (2) can be obtained by the acid-catalyzed reaction of a ketoester of formula (3) with a polyol of formula (4):

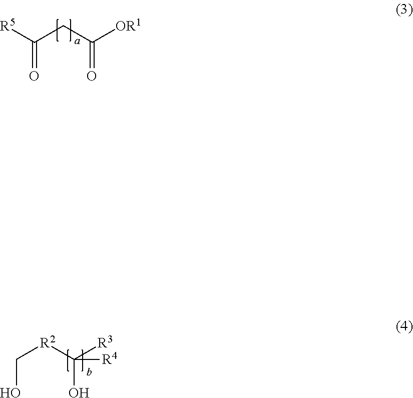

wherein R¹, R², R³, R⁴, R⁵, and a and b are as defined for formula (1). Reaction conditions for ketalization are described in WO 09/032905, for example. Alternatively, the ketal alcohol can be obtained by the acid-catalyzed reaction of a ketoalcohol of formula (5) with the polyol of formula (4)

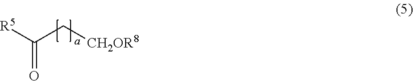

wherein R⁸ is hydrogen or an alcohol protecting group (which is subsequently removed), and R², R³, R⁴, and a and b are as defined in formula (1). Many of the compounds falling within the scope of formulae (2), (3), (4), and (5) can be bio-sourced. For example, levulinic acid is produced by the thermochemical treatment of various carbohydrates such as cellulose. Subsequent esterification with bio-sourced alkanols and ketalization of the resulting levulinate ester with polyhydroxy compounds such as glycerol or propylene glycol produces a levulinic ketal ester.

In a highly advantageous feature, the chemical and physical properties of the ketals can be adjusted to achieve the desired combination of properties, for example, solvent strength and volatility, by the choice of R¹, R², R³, R⁴, R⁶, and R⁷ groups and a and b in the ketals of formula (1). The ability to prepare ketals with a wide variety of R¹, R², R³, R⁴, R⁶, and R⁷ groups provides great flexibility in designing adjuvants that meet the technical requirements of biocide compositions.

Advantageously, the ketals of formula (1), and in particular (1a), (1b), (1c), (1d), have a volatility in a range that can be especially desirable for pesticide compositions such as emulsifiable concentrates, suspension concentrates, tank mix compositions, ready-to-use liquid compositions, and crop oil concentrates. Volatility manifests itself in a number of key properties, including boiling point, vapor pressure, relative evaporation rate, flash point, flammability, odor, and volatile organic compound (VOC) content. The desired volatility of an adjuvant varies considerably by application, and there are often conflicting considerations. For instance, highly volatile adjuvants evaporate quickly leaving behind residual biocide, but can also require special handling due to higher flammability. Appropriate selection of each of the specific R¹, R², R³, R⁴, R⁶, and R⁷ groups and a and b for the ketals of formula (1) can provide a desired volatility.

The adjuvant can advantageously serve as a solvent for the biocide, and can increase the efficacy of the biocide. The amount of adjuvant in the biocide composition depends upon the type of composition. Amounts of biocides useful for emulsifiable concentrates, suspension concentrates, tank mix compositions, and ready-to-use liquid compositions are provided below.

The adjuvants can be used with a wide variety of biocides. A biocide is a chemical substance capable of killing different forms of living organisms and used in fields such as medicine, agriculture, horticulture, forestry, and mosquito control. Biocides also include plant growth regulators. Biocides can be divided into two general sub-groups—pesticides and antimicrobials. The biocide can be a pesticide, which includes fungicides, herbicides, insecticides, fungicides, algicides, moluscicides, miticides, rodenticides, and combinations thereof. The pesticide can also include any compounds listed in The Pesticide Handbook, 14th edition, BCPC 2006. The biocide can be an antimicrobial, which includes germicides, antibiotics, bactericides, antivirals, antifungals, antiprotozoals, and antiparasites. In some embodiments, the biocide comprises a herbicide, an insecticide, a fungicide, a plant growth regulator, or a combination thereof. In some embodiments, the biocide comprises an insecticide.

The U.S. Environmental Protection Agency (EPA) defines a pesticide as “any substance or mixture of substances intended for preventing, destroying, repelling, or mitigating any pest”. A pesticide can be a chemical substance or biological agent (such as a virus or bacteria) used against pests, which include insects, plant pathogens, weeds, mollusks, birds, mammals, fish, nematodes (for example roundworms) and microbes that compete with humans for food, destroy property, spread disease, or are a general nuisance. In some embodiments, the biocide is a pesticide suitable for agricultural or horticultural use.

In some embodiments, the biocide is a herbicide. A herbicide is a pesticide that kills unwanted plants such as weeds. Herbicides can be selective, killing specific species of plants while leaving the desired crop relatively unharmed. Some herbicides act by interfering with the growth of weeds and are often based on plant hormones. Other herbicides are nonselective and kill all plants they come into contact with. Herbicides are widely used in agriculture and in landscaping, especially on grass turf. They are utilized in total vegetation control (TVC) programs for maintenance of highways and railroads. Smaller quantities are used in forestry, pastures, and wildlife habitat areas.

Chemical classes and specific examples of herbicides include: anilides such as propanil, aryloxycarboxylic acids such as MCPA-thioethyl; aryloxyphenoxypropionates such as clodinafoppropargyl, cyhalofopbutyl, diclofops, fluazifops, haloxyfops, quizalofops, chloroacetamides such as acetolochlor, alachlor, butachlor, dimethenamid, metolachlor, propachlor, cyclohexanedione oximes such as clethodim, sethoxydim, tralkoxydim; benzamides such as isoxaben; benzimidazoles such as dicamba, ethofumesate dinitroanilines such as trifluralin, pendimethalin; diphenyl ethers such as aclonifen, oxyfluorfen; glyphosate (a glycine derivative); hydroxybenzonitriles for example bromoxynil; imidazolinones such as fenamidone, imazapic, imazamox, imazapic, imazapyr, imazaquin; isoxazolidinones such as clomazone; paraquats such as bypyridylium; phenyl carbamates such as desmedipham, phenmedipham; phenylpyrazoles such as pyraflufen-ethyl; phenylpyrazolines such as pinoxaden, pyridinecarboxylic acids or synthetic auxins such as picloram, clopyralid, and triclopyr; pyrimidinyloxybenzoics such as bispyrtbac-sodium; sulfonylureas such as amidosulfuron, azimsulfuron, bensulfuron-methyl, chlorsulfuron, halosulfuron, flazasulfuron, foramsulfuron, flupyrsulfuron-methyl-sodium, nicosulfuron, rimsulfuron, sulfosulfuron, tribenuron-methyl, trifloxysurlfuron-sodium, triflusulfuron, tritosulfuron; triazolopyrimidines such as penoxsulam, metosulam, florasulam; triketones such as mesotriones, sulcotrione; ureas such as diuron, linuron; phenoxycarboxylic acids such as 2,4-D, MCPA, MCPB, mecoprops; triazines such as atrazine, simazine, terbuthylazine; fomesafen and combinations thereof.

In some embodiments, the biocide is an insecticide. An insecticide is a pesticide that kills insects in any developmental stage. Thus pesticides include ovicides and larvicides, which are used against the eggs and larvae of insects. Insecticides are used in agriculture, medicine, industry, and in the household.

Chemical classes and specific examples of insecticides include: Abamectin, emamectin, anthranilic diamides such as Rynaxypyr, synthetic auxins such as avermectin, amidines such as amitraz, carbamates such as aldicarb, carbofuran, carbaryl, methomyl, 2-(1-methylpropyl)phenyl methylcarbamate, chlorinated insecticides such as, for example, Camphechlor, DDT, hexachlorocyclohexane, gamma-hexachlorocyclohexane, Methoxychlor, pentachlorophenol, TDE, Aldrin, Chlordane, Chlordecone, Dieldrin, Endosulfan, Endrin, Heptachlor, Mirex, juvenile hormone mimics such as pyriproxyfen; neonicotinoids such as imidacloprid, clothianidin, thiacloprid, thiamethoxam, organophosphorus compounds such as acephate, azinphos-methyl, bensulide, chlorethoxyfos, chlorpyrifos, chlorpyriphos-methyl, diazinon, dichlorvos (DDVP), dicrotophos, dimethoate, disulfoton, dthoprop, fenamiphos, fenitrothion, fenthion, fosthiazate, malathion, methamidophos, methidathion, methyl-parathion, mevinphos, naled, omethoate, oxydemeton-methyl, parathion, phorate, phosalone, phosmet, phostebupirim, pirimiphos-methyl, profenofos, terbufos, tetrachlorvinphos, tribufos, trichlorfon, oxadiazines such as indoxacarb, plant toxin derived compounds such as derris (rotenone), pyrethrum, neem (azadirachtin), nicotine, caffeine; pheromones such as cuellure, methyl eugenol; pyrethroids such as, for example, Gamma Cyhalothrin, allethrin, bifenthrin, deltamethrin, permethrin, resmethrin, sumithrin, tetramethrin, tralomethrin, transfluthrin; selective feeding blockers such as flonicamid, pymetrozine, spinosyns such as spinosad; and combinations thereof.

In some embodiments, the biocide is a fungicide. Fungicides are chemical compounds used to prevent the spread of fungi in gardens and crops. Fungicides are also used to fight fungal infections. Fungicides can be contact or systemic. A contact fungicide kills fungi when sprayed on its surface. A systemic fungicide has to be absorbed by the fungus for the fungus to die.

Chemical classes and specific examples of fungicides include: aminopyrimidines such as bupirimate; anilinopyrimidines such as cyprodinil, mepanipyrim, pyrimethanil; heteroaromatics such as hymexazole; heteroaromatic hydrocarbons such as etridiazole; chlorophenyls/nitroanilines such as chloroneb, dicloran, quintozene, tecnazene, tolclofos-methyl; amides such as benzovindiflupyr, benzamide fungicides such as zoxamide; phenyl-benzamides such as benodanil, flutolanil and mepronil; pyridinyl-ethyl-benzamides such as fluopyram; phenyl-oxo-ethyl thiophene amides such as isofetamid; benzenesulfonamides such as flusulfamide; benzimidazoles such as acibenzolar, benomyl, benzothiazole, carbendazim, Albendazole, metrafenone, probenazole, thiabendazole, triazoxide, and benzimidazole precursor fungicides; carbamates such as propamocarb, diethofencarb; carboxamides such as diclocymet, ethaboxam, flutolanil; furan-carboxamides such as isotianil and fenfuram; oxathiin-carboxamides such as carboxin and oxycarboxin; thiazole-carboxamides such as thifluzamide; pyrazole-carboxamides such as bixafen, fluxapyroxad, furametpyr, isopyrazam, penflufen, penthiopyrad and sedaxane; pyridine-carboxamides such as boscalid; chloronitriles such chlorothalonil; cinnamic acid amides such as dimethomorph, flumorph; cyanoacetamide oximes such as cymoxanil, cyclopropancarboxamides such as carpropamid, dicarboximides such as iprodione, octhilinone, procymidone, vinclozolin; dimethyldithiocarbamates such as ferbam, metam, thiram, ziram, dinitroanilines such as fluazinam, dithiocarbamates such as mancopper, mancozeb, maneb, metiram, nabam, propineb, zineb; dithiolanes such as isoprothiolane; glucopyranosyl antibiotics such as streptomycin, validamycin; guanidines such as dodine, guazatine, iminoctadine, hexopyranosyl antibiotics such as kasugamycin; hydroxyanilides such as fenhexamid; imidazoles such as imazalil, oxpoconazole, pefurazoate, prochloraz, triflumizole; imidazolinones such as fenamidone; inorganics such as Bordeaux mixture, copper hydroxide, copper naphthenate, copper oleate, copper oxychloride, copper(II) sulfate, copper sulfate, copper (II) acetate, copper(II) carbonate, cuprous oxide, sulfur; isobenzofuranones such as phthalide; mandelamides such as mandipropamide; morpholines such as dodemorph, fenpropimorph, tridemorph, fenpropidin, piperalin, spiroxamine, aldimorph; organotins such as fentin; oxazolidinones such as oxadixyl; phenylamides such as benalaxyl, benalaxyl-M, furalaxyl, metalaxyl, metalaxyl-M, ofurace; phenylpyrazoles such as fipronil; phenylpyrroles such as fludioxonil; phenylureas such as pencycuron; phosphonates such as fosetyl; phthalamic acids such as tecloftalam; phthalimides such as captafol, captan, folpet; piperazines such as triforine; propionamides such as fenoxanil; pyridines such as pyrifenox; pyrimidines such as fenarimol, nuarimol; pyrroloquinolinones such as pyroquilon; qils such as cyazofamid; quinazolinones such as proquinazid; quinolines such as quinoxyfen; quinones such as dithianon; sulfamides such as tolylfluanid, dichlofluanid; strobilurines such as azoxystrobin, dimoxystrobin, famoxadone, fluoxastrobin, kresoxim-methyl, metominostrobin, picoxystrobin, pyraclostrobin, trifloxystrobin, orysastrobin, pyribencarb, pyrametostrobin, pyraoxystrobin; thiocarbamates such as methasulfocarb; thiophanates such as thiophanate-methyl; thiophencarboxamides such silthiofam; triazoles such as azaconazole, bitertanol, bromuconazole, cyproconazole, difenoconazole, diniconazole, epoxiconazole, fenbuconazole, fluquinconazole, flusilazole, flutriafol, fluotrimazole, hexaconazole, imibenconazole, ipconazole, metconazole, myclobutanil, penconazole, propiconazole, prothioconazole, simeconazole, tebuconazole, tetraconazole, triadimefon, triadimenol, triticonazole, quinconazole; triazolobenzothidazoles such as tricyclazole; valinamide carbamates such as iprovalicarb, benthiavalicarb; fluopicolide; pentachlorophenol; oomcyetes such as ametoctredin, fluopicolide, mandipropamid, amisulbrom, valifenalate and oxathiapiproline; flutianil; quinolines such as tebufloquin; pyrazolinones such as fenpyrazamine; and combinations thereof.

The biocide can be a plant growth regulator. Plant growth regulators include plant hormones (also known as phytohormones), which are chemicals that regulate plant growth. Plant hormones are signal molecules produced within the plant, and occur in extremely low concentrations. Hormones regulate cellular processes in targeted cells locally, and when moved to other locations, in other locations of the plant. Plants, unlike animals, lack glands that produce and secrete hormones. Plant hormones shape the plant, affecting seed growth, time of flowering, the sex of flowers, and senescence of leaves and fruits. They affect which tissues grow upward and which grow downward, leaf formation and stem growth, fruit development and ripening, plant longevity, and even plant death. Hormones are vital to plant growth and lacking them, plants would be mostly a mass of undifferentiated cells.

Chemical classes and specific examples of plant growth regulators include: aviglycine; cyanamide; gibberellins such gibberellic acid; quaternary ammoniums salts such as chlormequat chloride, mepiquat chloride, ethylene generators such ethephone; and combinations thereof.

The biocide can be a rodenticide. Rodenticides are pesticides that kill rodents. Rodents are difficult to kill with poisons because their feeding habits reflect their role as scavengers. Rodents tend to eat a small bit of something and wait, and if they do not get sick, they continue eating. An effective rodenticide must be tasteless and odorless at lethal concentrations, and have a delayed effect. Rodenticides include anticoagulants. Anticoagulants are defined as chronic (death occurs after 1 to 2 weeks post ingestion of the lethal dose, rarely sooner), and as single dose (second generation) or multiple dose (first generation) cumulative rodenticides. Fatal internal bleeding is caused by lethal doses of anticoagulants.

Chemical classes and specific examples of rodenticides include 4-hydroxycoumarins such as warfarin, coumatetralyl, difenacoum; indandiones such as pindone, diphacinone, chlorophacinone, brodifacoum, bromadiolone and flocoumafen; 4-hydroxy-1-benzothiin-2-one (4-hydroxy-1-thiacoumarin); difethialone; metal phoshides such as zinc phosphide, aluminum phosphide, calcium phosphide, magnesium phosphide; calciferols (vitamins D), such as cholecalciferol (Vitamin D₃), ergocalciferol (vitamin D₂); and combinations thereof.

Miticides are pesticides that kill mites. Miticides include antibiotic miticides, carbamates, formamidines, mite growth regulators, organochlorine compounds, permethrin, and organophosphates. Molluscicides are pesticides that kill mollusks, such as slugs and snails. Molluscicides include metaldehyde, methiocarb, aluminium sulfate, and combinations thereof.

Nematicides are pesticides that kill parasitic nematodes (a phylum of worm). One nematicide is the residue of neem seeds after oil extraction.

In some embodiments, the biocide is an antimicrobial. Antimicrobials include antibiotics, bactericides, antivirals, antifungals, antiprotozoals, and antiparasites. Such compounds, including bactericides, include active chlorine compounds such as hypochlorites, chloramines, dichloroisocyanurate, trichloroisocyanurate, wet chlorine, chlorine dioxide; active oxygen compounds (peroxides) such as peracetic acid, potassium persulfate, sodium perborate, sodium percarbonate, and urea perhydrate; iodine compounds such as iodpovidone, povidone-iodine, betadine, Lugol's solution, iodine tincture, iodinated nonionic surfactants; concentrated alcohols such as ethanol, 1-propanol, 2-propanol, 2-phenoxyethanol, 1- and 2-phenoxypropanols; phenols such as phenol (“carbolic acid”), cresols, “Lysole” (cresol in combination with liquid potassium soaps), halogenated phenols (chlorinated, brominated) such as hexachlorophene, triclosan, trichlorophenol, tribromophenol, pentachlorophenol, Dibromol, and salts thereof; quaternary ammonium salts such as benzalkonium chloride, cetyl trimethylammonium bromide or chloride, didecyldimethylammonium chloride, cetylpyridinium chloride, benzethonium chloride; other nitrogen-containing compounds such as chlorhexidine, glucoprotamine, octenidine dihydrochloride; strong oxidizers such as ozone and permanganate; heavy metals and their salts such as colloidal silver, silver nitrate, mercury chloride, phenylmercury salts, copper sulfate; strong acids such as phosphoric acid, nitric acid, sulfuric acid, amidosulfuric acid, toluenesulfonic acid; alkalis such as sodium, potassium, and calcium hydroxides; and combinations thereof.

Antiseptics are antimicrobials that are applied to living tissue/skin to reduce the possibility of infection, sepsis, or putrefaction. Antiseptics are generally distinguished from antibiotics by the latter's ability to be transported through the lymphatic system to destroy bacteria within the body, and from disinfectants, which destroy microorganisms found on non-living objects.

Antibiotics include bactericide antibiotics which kill bacteria; and bacteriostatic antibiotics which only slow down the growth or reproduction of bacteria. Examples of antibiotics include penicillin, cephalosporins, aminoglycosides, fluoroquinolones, nitrofurans, vancomycin, monobactams, co-trimoxazole, and metronidazole, azoxystrobin.

The amount of biocide in the composition depends upon the type of composition. Amounts of biocides useful for emulsifiable concentrates, suspension concentrates, tank mix composition, and ready-to-use liquid composition are provided below.

In some embodiments, the biocide composition further comprises an oil. The oil can serve as a diluent, solvent, penetrant, and can have surfactant properties as well. The oil can increase the efficacy of the biocide, improve spray wetting of surfaces, reduce evaporation, increase resistance to wash-off, and in particular, increase penetration of a herbicide through the cuticle of a plant.

Chemical classes and specific examples of oils include: Guerbet alcohols derived from fatty alcohols having 6 to 18, specifically 8 to 10, carbon atoms; esters of linear C₆-C₂₂ fatty acids with linear or branched C₆-C₂₂ fatty alcohols such as myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate; esters of branched C₆-C₁₃ carboxylic acids with linear or branched C₆-C₂₂ fatty alcohols; esters of linear C₆-C₂₂ fatty acids with branched alcohols such as 2-ethylhexanol; esters of C₁₈-C₃₈ hydroxyalkyl carboxylic acids with linear or branched C₆-C₂₂ fatty alcohols such as dioctyl malate, esters of linear and/or branched fatty acids with polyhydric alcohols (for example, propylene glycol, dimerdiol or trimertriol) and/or Guerbet alcohols; triglycerides of C₆-C₁₀ fatty acids; liquid mono-/di-/triglycerides of C6-C18 fatty acids; esters of C₆-C₂₂ fatty alcohols and/or Guerbet alcohols with aromatic carboxylic acids (for example benzoic acid); esters of C₂-C₁₂ dicarboxylic acids with C₁-C₂₂ linear or branched alcohols such as dibutyl adipate; esters of C₂-C₁₂ dicarboxylic acids with C₂-C₁₀ polyols having 2 to 6 hydroxyl groups; vegetable oils and synthetic esters of vegetable oils; branched primary alcohols; substituted cyclohexanes such as dialkylcyclohexanes; carbonates of linear and branched C₆-C₂₂ fatty alcohols, such as dicaprylyl carbonate; carbonates of Guerbet alcohols; carbonates of C₆-C₁₈ fatty alcohols, specifically C₈-C₁₀ fatty alcohols; esters of benzoic acid with linear and/or branched C₆-C₂₂ alcohols, such as CETIOL™ AB (C₁₂-C₁₅ alkyl benzoate); linear or branched, symmetrical or asymmetrical di(C₆-C₂₂ alkyl) ethers such as dicaprylyl ether; ring-opening products of epoxidized fatty acid esters with polyols; silicone oils such as cyclomethicones, silicone methicones; aliphatic or naphthenic hydrocarbons; terpenes and terpene derivatives such as squalane and squalene; mineral oils; alkoxylated fatty acid esters, and combinations thereof.

The oil can be an alkoxylated fatty acid ester, including alkoxylated fatty acid glycerides (also known as alkoxylated triglycerides) and are often termed “semi-natural” surfactants as they are made by alkoxylation (ethoxylation or propoxylation) of fatty acid esters of natural origin such as vegetable oil (e.g. a seed oil). Examples of alkoxylated fatty acid esters prepared from vegetable oils include ethoxylated fatty acid esters containing 10 to 60 ethylene oxide units. Exemplary are POE 25 castor oil, POE 30 soybean oil, and POE 30 rapeseed oil, where “POE” stands for polyoxyethylene and the number denotes the average number of polyoxyethylene units.

In some embodiments, the oil is a fatty acid ester of a C₁₋₄ alcohol, an alkoxylated fatty acid ester, a vegetable oil, a mineral oil, or a combination thereof. The amount of oil in the biocide composition depends upon the type of composition. Amounts of oils useful for emulsifiable concentrates, suspension concentrates, tank mix compositions, ready-to-use liquid compositions, and crop oil compositions are provided below.

In some embodiments, the biocide composition further comprises a solvent. The solvent can serve as a diluent, and to dissolve or partially dissolve the biocide. The solvent can comprise an aliphatic hydrocarbon, a ketone, an alcohol, an ester, an amide, an ether, or a combination thereof. The aliphatic hydrocarbon can comprise linear or branched alkenes (isoparaffins), alkenes (olefins), and cyclic alkanes (naphthenes). In some embodiments, the solvent comprises isoparaffins.

Although the biocide composition can be applied directly to a pest, for example insects or weeds, or their environment, for example crops or turf, the biocide composition can also be first diluted with water, and then the target pest or its environment is contacted with an effective amount of the diluted composition to control the pest. Thus, in some embodiments, the biocide composition further comprises water, the water serving as a diluent for a suspension concentrate or an emulsifiable concentrate comprising the biocide and the adjuvant. The resulting diluted composition is sometimes referred to as a “tank mix composition”. When the biocide is a liquid, or a solid that completely dissolves in the adjuvant and the optional oil, the biocide composition is sometimes referred to as an “emulsifiable concentrate”. The emulsifiable concentrate can be diluted with water to form an emulsion using high-speed mixing. When the biocide composition is an emulsifiable concentrate, the composition diluted with water is an emulsion of droplets of the adjuvant and optional oil in water. When the biocide is a solid that is insoluble or only partially soluble in the adjuvant and the optional oil, the biocide composition is sometimes referred to as a “suspension concentrate”. After mixing with water, the resulting diluted composition comprises a suspension of solid particles of the biocide as well as an emulsion of droplets of the adjuvant and optional oil. For both the emulsifiable concentrate and the suspension concentrate, the diluted composition is sometimes referred to as a “tank mix composition”.

The tank mix composition can be applied to a pest or its environment by a variety of means including spraying, atomizing, dispersing, or pouring. The method used will depend on the particular objectives and circumstances of the biocide use, and can be readily determined by the end user.

A surfactant can aid in the formation of stable suspension concentrates, and in the formation of stable tank mix compositions from emulsifiable concentrates and suspension concentrates. Stability is defined as the ability of the composition to resist agglomeration and/or coalescence of the dispersed solid and/or liquid particles and to resist the settling out of the particles from the continuous aqueous phase. Thus, in some embodiments, the biocide composition further comprises a surfactant. Surfactants (also known as “surface-active agents”) generally modify, and most often reduce, the surface tension of a liquid. Surfactants comprise both hydrophilic and hydrophobic (lipophilic) groups. Depending on the nature and relative amounts of the hydrophilic and lipophilic groups in a surfactant molecule, surfactants can serve as wetting agents, dispersing agents (i.e. dispersants), emulsifiers, anti-foaming agents (i.e. defoamers), or a combination thereof. The terms “dispersing agent” and “dispersant” as used herein relate to surfactants that are effective in dispersion of solid particles in an aqueous medium. The terms “emulsifying agent” and “emulsifier” as used herein relate surfactants that are effective in dispersion of liquid immiscible oil droplets in an aqueous medium. Thus the surfactant comprises an emulsifier, a dispersant, or a combination thereof, and can serve to emulsify or disperse the biocide in water. The surfactant can comprise a nonionic surfactant, an anionic surfactant, a cationic surfactant, or a combination thereof. Surfactants are classified as anionic, non-ionic, or cationic based on the polarity of their hydrophilic groups. Anionic surfactants have negatively charged hydrophilic groups, cationic surfactants have positively charged hydrophilic groups, and nonionic surfactants have polar, but uncharged, hydrophilic groups. Commercially available surfactants are described in McCutcheon's Detergents and Emulsifiers Annual, Allured Publishing Corp., Ridgewood, N.J., as well as in Sisely and Wood, Encyclopedia of Surface Active Agents, Chemical Publishing Co., Inc., New York, 1964.

The surfactant can be an anionic surfactant. An anionic surfactant is a surfactant in which functional groups in the hydrophilic portion of the molecule ionize to form anionic functional groups when mixed with water. Anionic functional groups of anionic surfactants include carboxylate, sulfate, sulfonate, and phosphate anions, formed by ionization of the respective conjugate acids. Examples of anionic surfactants include sodium alkylnaphthalene sulfonates, naphthalenesulfonates, sulfonated formaldehyde condensates, alkylbenzenesulfonates, lignin sulfonates, alkyl sulfates, alkyl ether sulfates, dialkyl sulfosuccinates, N,N-dialkyltaurates, polycarboxylates, phosphate esters, ethoxylated tristyrylphenol phosphate salts, alkali salts of fatty acids; and combinations thereof.

The surfactant can be a nonionic surfactant. A nonionic surfactant does not contain ionizable groups, but the hydrophilic group is polar. Examples of nonionic surfactants include ethoxylated alcohols, ethoxylated alkylphenols, ethoxylated sorbitol esters, ethoxylated fatty acid esters, polyoxyethylene/polyoxypropylene block copolymers, glycerol esters, and alkylpolyglycosides where the number of glucose units, referred to as degree of polymerization (D.P.), can be 1 to 3 and the alkyl units can be C6-C14.

The term “ethoxylated” refers to the presence of polyether chains comprising one or more divalent oxyethylene units (—OCH₂CH₂—) formed by reaction of ethylene oxide with hydroxyl groups of an alcohol or polyol, for example, sorbitan, sorbitol, or a fatty acid, respectively. Similarly, the term “alkoxylated” refers to the presence of polyether chains comprising one or more divalent oxyalkylene units (—OCₙH₂ₙ—), where n is 2, 3, or 4, formed by reaction of ethylene oxide, propylene oxide, butylene oxide, or combinations thereof, with hydroxyl groups of an alcohol or polyol. Any hydroxyl groups present after alkoxylation can be esterified. The term “polyoxyalkylene”, for example “polyoxyethylene”, “polyoxypropylene”, and “polyoxybutylene”, can be used to name an alkoxylated surfactant. The average number of oxyalkylene units per surfactant molecule can be included in the name.

The surfactant can be a cationic surfactant. An cationic surfactant is a surfactant in which functional groups in the hydrophilic portion of the molecule ionize to form cationic functional groups when mixed with water. Examples of cationic surfactants include quaternary ammonium salts such benzylalkylammonium salts, pyridinium salts, quaternary imidazolium compounds, and combinations thereof. The cationic surfactant can also be a protonated amine such as ammonium salts of N-oleyl-1,3-diaminopropane, oleylamine, lauryl dimethylamine, and combinations thereof.

Without being bound by theory, surfactants function by reducing surface tension. The ability of surfactants to reduce surface tension depends upon the molecular structure of the surfactant. In particular, the hydrophilic-lipophilic balance (HLB) determines whether the surfactant is soluble in water and whether water-immiscible liquid droplets can be stabilized (i.e. emulsified) in water. The HLB value of a surfactant indicates the overall polarity of the molecule, and is in the range of 1 to 40, with the most common commercial surfactants having an HLB value of 1 to 20. The HLB value increases with increasing hydrophilicity. Surfactants with HLB values of 0 to 7 are considered lipophilic, surfactants with HLB values of 12 to 20 are considered hydrophilic, and surfactants with HLB values of 7 to 12 are considered intermediate.

Examples of hydrophilic surfactants are anionic surfactants such as the sodium, calcium, and isopropylammonium salts of branched or linear alkylbenzenesulfonates. Non-ionic surfactants such as ethoxylated castor oil, ethoxylated sorbitan oleates, ethoxylated alkyl phenols, and ethoxylated fatty acids can have an intermediate HLB value which depends upon chain length and degree of ethoxylation. Triesters of oleic acid and sorbitan (e.g. sorbitan trioleate) and triesters of stearic acid and sorbitan (e.g. sorbitan tristearate) are examples of lipophilic, nonionic surfactants. Lists of surfactants and their HLB values have been published widely, for example in A.W. Adamson, Physical Chemistry of Surfaces, John Wiley and Sons, 1982.

The biocide composition can be an emulsifiable concentrate comprising the biocide, the adjuvant, the surfactant, and optionally the oil. In the emulsifiable concentrate, the biocide is dissolved in the adjuvant and optionally the oil. The emulsifiable concentrate can form an oil-in-water emulsion when diluted with water.

In a suspension concentrate, the biocide is present as solid particles suspended in the liquid phase comprising the adjuvant and optionally the oil. When solids in the particulate phase come close to each other and their mutual attraction overcomes repulsive forces, recombination can occur in which the particles stick together either by flocculation or by agglomeration. Dispersants can absorb onto the particle surface to create an electrostatic and/or steric barrier between the particles, thus reducing particle-to-particle attractive forces and thereby stabilizing the suspension. Therefore the biocide composition can advantageously comprise a surfactant, in which the surfactant is effective in dispersing particles of solid biocide in the aqueous phase. The dispersant can be present in an amount of 1 to 15 weight %, specifically 1 to 10 weight %, more specifically 1 to 5 weight %, based on the total weight of the composition.

Examples of dispersing agents include anionic surfactants such as phosphate esters of tristyrylphenol ethoxylates (e.g. SOPROPHOR™ 3D33), alkylarylsulfonic acids and their salts (e.g. SUPRAGIL™ MNS90), lignin sulfonates (e.g. ammonium lignosulfonate or sodium lignosulfonate), polyphenol sulfonates, polyacrylic acids and acrylic graft copolymers such as acrylic acid/methyl methacrylate/polyethylene glycol graft copolymers and their salts (e.g. ATLOX™ 4913), phospholipids such as lecithin; non-ionic surfactants such as fatty alcohol ethers, polyoxyethylene/polyoxypropylene block copolymers (e.g. PLURONIC™ F108 polyoxyethylene/polyoxypropylene block copolymer) and other polyoxyalkylene-containing polymers such as ATLOX™ 4912 (block copolymer of poly(ethylene glycol) and hydroxystearic acid), ATLAS™ G-5000 (poly(alkylene glycol)ether), ethoxylated amides, fatty acid alkanol amides; and combinations thereof.

In addition to solid particles suspended in the aqueous phase of the suspension composition, liquid droplets comprising the adjuvant and optional oil can be emulsified in the aqueous phase. In some embodiments, an emulsifier is needed to maintain the adjuvant and optional oil in suspension as finely dispersed droplets. In these compositions the surfactant serving to disperse the particles of solid bioicide can also be effective is emulsifying the adjuvant and optional oil, i.e. the surfactant can have both dispersing and emulsifying properties. However, in some embodiments, an oil which has self-emulsifying properties is present. For example when the oil is an ethoxylated fatty acid ester such as ethoxylated soybean oil (POE 20-30), it can serve as the emulsifier, and then the surfactant (dispsersant) does not need to have an emulsifying property to provide a stable suspension concentrate. Thus in a suspension concentrate, the surfactant can have a dispersing property and optionally an emulsifying property.

Surfactants that are useful as emulsifiers generally reside at the oil-water interface with their lipophilic portion immersed in the water-immiscible liquid droplets (oil phase) and their hydrophilic portion penetrating the surrounding aqueous phase, thereby reducing the surface tension between the two phases. Emulsifiers can prevent the coalescence of water-immiscible liquid droplets in water and thus help maintain stable dispersions of the water-immiscible liquid droplets in water, which are known as emulsions.

The emulsifier can facilitate the formation of emulsions of droplets comprising the adjuvant and optional oil in the continuous aqueous phase. The presence of solid particles of the biocide and any other water-insoluble components can significantly influence the effectiveness of an emulsifier. Stable emulsions can be obtained by empirically matching the HLB value of the emulsifier to the adjuvant, the optional oil, and the dispersed solid particles in the composition. The emulsifier can promote dispersion of the suspension concentrate composition when it is diluted with water, for example in forming a tank mix composition prior to spray application. In a suspension concentrate, the emulsifier can be present in an amount of 0 to 20 weight %, specifically 2 to 10 weight %, more specifically 3 to 7 weight %, based on the total weight of the composition. The total amount of surfactant, including dispersant and emulsifier, can be 1 to 35 weight %, specifically 2 to 20 weight %, more specifically 3 to 12 weight %, based on the total weight of the composition.

In an emulsifiable concentrate, in which there are no undissolved solid biocide particles, an emulsifier can be used to facilitate emulsification of the concentrate when mixed with water, and to stabilize the resulting emulsion (tank mix composition) against coalescence and phase separation. In an emulsifiable concentrate, the emulsifier can be present in an amount of 0.1 to 15 weight %, specifically 1 to 10 weight %, more specifically 1 to 5 weight %, based on the total weight of the composition.

For reasons including favorable physical properties, commercial availability, and cost, anionic surfactants including linear (unbranched) alkylbenzenesulfonates and branched alkylbenzenesulfonates, specifically linear alkylbenzenesulfonates can be used. Exemplary linear alkylbenzenesulfonates include dodecylbenzenesulfonates, for example, calcium dodecylbenzenesulfonate, e.g. RHODACAL™ 70/B (Rhodia) or PHENYLSUFLONAT™ CA100 (Clariant), and isopropylammonium dodecylbenzenesulfonate, e.g. ATLOX™ 3300B (Croda).

For reasons including favorable physical properties, commercial availability, and cost, non-ionic surfactants including sorbitan esters, ethoxylated sorbitan esters, sorbitol esters, ethoxylated sorbitol esters, ethoxylated fatty acid esters (including ethoxylated triglycerides), and combinations thereof can be used. Examples of ethoxylated sorbitan esters include ethoxylated sorbitan oleate (e.g. monooleate, trioleate) and ethoxylated sorbitan laurate (e.g. trilaurate), each having 10-30 oxyethylene units (i.e. POE 10-30). Examples of ethoxylated sorbitol esters include ethoxylated sorbitol oleate (e.g. hexaoleate) and ethoxylated sorbitol laurate (e.g. hexalaurate). Examples of ethoxylated fatty acid esters include ethoxylated seed oils such as ethoxylated soybean oil, ethoxylated castor oil, and ethoxylated rapeseed oil, each having 10-30 oxyethylene units (i.e. POE 10-30). Specific examples of non-ionic surfactants include POE 20 sorbitan monooleate (EMSORB™ 6900, available from Cognis and TWEEN™ 80, available from Croda), POE 40 sorbitol hexaoleate (CIRRESOL™ G-1086, available from Croda, POE 30 ethoxylated soybean oil (AGNIQUE™ SBO-30, available from Cognis), and POE 25 ethoxylated castor oil (TRYLOX™ 5904, available from Cognis).

A surfactant can partition between the aqueous phase and the water-immiscible phase comprising the adjuvant and the optional oil. For example, a dispersant can reside in the aqueous phase where it facilitates dispersion of the solid particles of the biocide in the aqueous phase, and an emulsifier can reside in the water-immiscible liquid phase where it facilitates formation of an emulsion of the water-immiscible liquid phase in the aqueous phase. One skilled in the art will appreciate that while the major portion of a component of a dispersion or emulsion can reside in a particular phase, lesser amounts can be present in other phases as well. Thus while the major portion of an emulsifier can be present in the water-immiscible liquid phase rather than the aqueous phase, its emulsifying effect stems from its presence at the interface of the water-immiscible liquid phase droplets and the aqueous phase. Moreover, depending upon its HLB value, a significant amount of the emulsifier can be present in the aqueous phase as well. Also, while the major amount of a dispersant can reside in the aqueous phase, depending upon its HLB value, a significant amount of dispersant can be present in the water-immiscible liquid phase as well. Moreover, minor amounts of water (less than 2% by weight at 20° C.) can be present in the water-immiscible phase, and minor amounts of water-immiscible components (less than 2% by weight at 20° C.) can be present in the aqueous phase.

The adjuvant can also partition between the aqueous phase and the water-immiscible phase, or at the interface between a continuous phase and a disperse phase. While the major portion of the adjuvant can reside in a particular phase, lesser amounts can be present in other phases as well. Thus in some embodiments, the adjuvant is present in the aqueous phase, the water-immiscible liquid phase, or a combination thereof. For example, while the major portion of the adjuvant can be present in the water-immiscible liquid phase rather than the aqueous phase, a significant amount of the adjuvant can be present in the aqueous phase as well.

In some embodiments, the biocide composition further comprises a penetrant. Penetrants are substances which are customarily used to promote penetration of herbicides into plants. Penetrants have the ability to penetrate into the cuticle of the plant from the aqueous spray liquor and/or from the spray covering, and thereby increase the penetration of the biocide into the cuticle of the plant. The method described in the literature (Baur et al., Pesticide Science 51, 131-152, 1997) can be used for determining this property.

In some embodiments, the penetrant is an alkanol alkoxylate of formula (10)

R⁸—O-(AO)ₘ—R⁹ (10)

wherein R⁸ is a straight chain or branched alkyl group having 4 to 20 carbon atoms; R⁹ is methyl, ethyl, n-propyl, isopropyl, n-butyl, isobutyl, t-butyl, n-pentyl, or n-hexyl; AO is an ethylene oxide radical, a propylene oxide radical, a butylene oxide radical, or a combination of an ethylene oxide and propylene oxide radical, or an ethylene oxide radical and a butylene oxide radical; and m is 2 to 30.

Other examples of the oil and the surfactant, as described above, can also serve as penetrants.

In some embodiments, the biocide composition further comprises an additive. The additive can be a wetting agent, an anti-freeze, a preservative, a stabilizing agent, a buffer, a rheology modifier, an anti-foam agent, a crystallization inhibitor, a fertilizer, or a combination thereof

The biocide composition can be an emulsifiable concentrate, a suspension concentrate, a suspo-emulsion concentrate, a tank mix composition, or a ready-to-use liquid composition.

In some embodiments, the biocide composition is an emulsifiable concentrate comprising the biocide, the adjuvant, the surfactant, and optionally the oil. Prior to dilution with water, the emulsifiable concentrate can comprise, based on the total weight of the composition, 0.01 to 99 weight %, specifically 0.05 to 75 weight %, more specifically 0.1 to 40 weight % of the biocide; or 0.01 to 10 weight %, specifically 0.05 to 5 weight %, more specifically 0.1 to 5 weight %. Prior to dilution with water, the emulsifiable concentrate can also comprise, based on the total weight of the composition, 0.01 to 99 weight %, specifically 0.05 to 75 weight %, more specifically 0.1 to 40 weight % of the biocide; 0.1 to 99 weight %, specifically 3 to 80 weight %, more specifically 10 to 70 weight % of the adjuvant; 0.1 to 15 weight %, specifically 1 to 10 weight %, more specifically 1 to 5 weight % of the surfactant; and 0 to 50 weight %, specifically 5 to 30 weight %, more specifically 10 to 25 weight % of the oil.

In some embodiments, the biocide composition is a suspension concentrate comprising the biocide, the adjuvant, water, the surfactant, and optionally the oil. The suspension concentrate can comprise, based on the total weight of the composition, 0.1 to 90 weight %, specifically 1 to 60 weight %, more specifically 10 to 50 weight % of the biocide; 5 to 60 weight %, specifically 10 to 50 weight %, more specifically 20 to 40 weight % of the adjuvant; 20 to 60 weight %, specifically 20 to 60 weight %, more specifically 20 to 50 weight % of water; 1 to 35 weight %, specifically 2 to 20 weight %, more specifically 3 to 12 weight % of the surfactant; and 0 to 70 weight %, specifically 10 to 60 weight %, more specifically 20 to 50 weight % of the oil.

The suspension concentrate can be prepared in two steps. First, an aqueous suspension of the biocide and the surfactant (dispersant), but not the adjuvant or the optional oil, in water is first prepared. The aqueous suspension can be prepared by ball-milling, bead-milling, sand-milling, colloid-milling, or air-milling, optionally in combination with high-speed blending under high shear. Then the water-immiscible phase, including the adjuvant, and optionally an emulsifying surfactant and an optional oil, is added to the aqueous suspension using high-speed blending at high shear to form the suspension concentrate. The suspension concentrate comprises a suspension of solid biocide particles and an emulsion of water-immiscible liquid droplets in the aqueous phase. The aqueous phase thus functions as a continuous liquid medium for both the dispersed solid particles of the biocide and also the emulsified liquid droplets comprising the adjuvant and optionally the oil. For stable suspension concentrates, the average particle size of the dispersed solid biocide particles and the emulsified droplets is less than 10 μm.

In some embodiments, the biocide composition is a tank mix composition comprising the biocide, the adjuvant, water, the surfactant, and optionally the oil. The tank mix composition can comprise, based on the total weight of the composition, 0.0005 to 80 weight %, specifically 0.002 to 40 weight %, more specifically 0.005 to 8 weight % of the adjuvant; or 0.0005 to 20 weight %, specifically 0.002 to 10 weight %, more specifically 0.005 to 2 weight % of the adjuvant. The tank mix composition can also comprise, based on the total weight of the composition, 0.0005 to 20 weight %, specifically 0.002 to 10 weight %, more specifically 0.005 to 2 weight % of the biocide; 0.0005 to 80 weight %, specifically 0.002 to 40 weight %, more specifically 0.005 to 8 weight % of the adjuvant; 40 to 99 weight %, specifically 50 to 90 weight %, more specifically 50 to 80 weight % water; 0.1 to 30 weight %, specifically 1 to 25 weight %, more specifically 5 to 20 weight % of the surfactant; and 0 to 20 weight %, specifically 1 to 15 weight %, more specifically 1 to 10 weight % of the oil.

In some embodiments, the biocide composition is a ready-to-use liquid composition comprising the biocide, the adjuvant, a solvent other than the adjuvant, and optionally a thickening agent, a propellant, an attractant, or a combination thereof; wherein the solvent is capable of 90% vaporization in 5 minutes.

The thickening agent serves to increases the viscosity of the composition so that the composition is capable of adhering to a vertical surface such that that none of the ready-to-use liquid composition dislodges from the vertical surface for 72 hours after application and evaporation of the volatile solvents. Natural or synthetic polysaccharide gums and clays can be used as thickening agents. Examples of polysaccharide thickening agents include xanthan gum, guar gum, gum arabic, alginin, gum tragacanth, sodium alginate, and combinations thereof. In some embodiments, organically modified bentonite clay is used as the thickening agent. The composition comprises 0 to 5 weight %, specifically 0.1 to 1 weight %, more specifically 0.5 to 1 weight % of the thickening agent. The composition can include combinations of thickening agents.

The ready-to-use liquid composition comprises a solvent other than the adjuvant. The solvent can serve as a diluent, and to dissolve or partially dissolve the biocide. The solvent can comprise an aliphatic hydrocarbon, a ketone, an alcohol, an ester, an amide, an ether, or a combination thereof. The aliphatic hydrocarbon can comprise linear or branched alkenes (isoparaffins), alkenes (olefins), and cyclic alkanes (naphthenes). In some embodiments, the solvent comprises isoparaffins. The solvent used in the ready-to-use liquid composition is volatile so that it vaporizes relatively quickly after being dispensed from a pressurized container. It is desirable to limit the amount of certain solvents in the composition and, in particular, to limit the amount of solvents that are classified as volatile organic compounds (“VOC's”). Accordingly, it is desirable that the amount of VOC's in the composition be below governmental limits such as less than 15 weight % (e.g. 1 to 15 weight %).

After packaging, the ready-to-use liquid composition can be applied as an aerosol. In order to form an aerosol, the composition can comprise a propellant, which pressurizes the storage container and which creates the aerosol upon release of the composition from the container. Examples of propellants include propane, isobutane, dimethyl ether, difluoroethane, tetrafluoroethane, carbon dioxide, and combinations thereof.

The ready-to-use liquid composition can optionally comprise an attractant.

The term “attractant” refers to any material that causes a pest or pest population to be drawn to it or, drawn to a location in which the attractant is dispersed at a high frequency than the frequency at which the pests are drawn to the location in the absence of the attractant. The particular attractant used can vary depending on the pest targeted for control.

The ready-to-use liquid composition can comprise, based on the total weight of the composition, 0.0005 to 2 weight %, specifically 0.002 to 1 weight %, more specifically 0.005 to 0.5 weight percent of the biocide; 10 to 60 weight %, specifically 20 to 50 weight %, more specifically 30 to 40 weight % of the adjuvant; 10 to 60 weight %, specifically 20 to 50 weight %, more specifically 30 to 40 weight % of the solvent; 0 to 5 weight %, specifically 0.1 to 1 weight %, more specifically 0.5 to 1 weight % of the thickening agent; 0 to 75 weight %, specifically 5 to 50 weight %, more specifically 10 to 30 weight % of the propellant; and 0 to 70 weight %, specifically 5 to 60 weight %, more specifically 10 to 40 weight % of the attractant.

The ready-to-use liquid composition can be applied to a target void, crevice, space, or, surface. Once dispensed from its storage container, the solvent vaporizes leaving behind residual biocide. Depending upon the volatility and amount of solvents, the solvent either evaporates after application to the target surface, or evaporates from the aerosol such that dried biocide contacts the target surface. In some embodiments, the solvent is 90% vaporized within 5 minutes, specifically within 1 minute, more specifically within 30 seconds, and still more specifically within 5 seconds of application of the composition. In some embodiments, the solvent is 90% vaporized prior to the composition contacting the target surface.

The biocide can be supplied to the end-user as a liquid concentrate, such as a an emulsifiable concentrate, a suspension concentrate, a suspo-emulsion concentrate, or a solution; or as a dry powder, such as a wettable powder, a dry flowable powder, or a soluble powder. The end-user then dilutes the concentrate or powder with water to form a tank mix composition having the desired biocide concentration. Advantageously, the end-user can also mix various additives with the concentrate or powder and water. For example the additive can be a crop oil concentrate comprising the adjuvant. The crop oil concentrate can serve to enhance the physical properties of the tank mix composition. For example, the crop oil concentrate can enhance the effectiveness of the biocide by increasing penetration and absorption of the biocide into the target organism. The crop oil concentrate can also enhance wetting of surfaces by the composition, enhance adhesion of the composition to surfaces, and increase the aerosol droplet size, thereby reducing drift of the aerosol away from the target area. The crop oil concentrate can include for example, plant based oils, petroleum based oils, surfactants, silicones, nutrient materials, agents to control spray drift, or other adjuvants that can be added to a tank mix composition.

In some embodiments, the crop oil concentrate further comprises a surfactant, and optionally an oil. The oil can comprise a fatty acid ester of a C₁₋₄ alcohol, an aliphatic carboxylic acid, or a combination thereof. The crop oil concentrate can comprise, based on the total weight of the composition, 2 to 95 weight %, specifically 10 to 90 weight %, more specifically 20 to 80 weight % of the adjuvant; 2 to 40 weight %, specifically 2 to 20 weight %, more specifically 2 to 10 weight % of the surfactant; and 0 to 95 weight %, specifically 5 to 90 weight %, more specifically 10 to 80 weight % of the oil.

Further disclosed herein is a method of controlling a pest, the method comprising contacting the pest or the locus of the pest with any of the above-described compositions, in an amount effective to control the pest. Where the composition is supplied as a concentrate, the concentrate is diluted prior to contacting as described above. The locus of the pest includes any part of its environment, for example the leaves, stems, or seeds of a plant, the soil of a plant, the habitat of a rodent, and the like. Amounts effective to control the pest will depend on the particular biocide used, and can be determined by one of ordinary skill in the art without undue experimentation.

Seeds can be treated with biocides to reduce yield losses during cultivation and for enhancing the agronomic and nutritional value of the produce. The biocide can include any of the biocides disclosed herein, for example fungicides, insecticides, rodenticides, nematocides, miticides, and bird repellents. Thus in an embodiment, a seed treatment composition comprises the biocide and the adjuvant. The treatment composition can comprise a polymeric binder, water, or a combination thereof. For example, the biocide can be formulated in a coating composition to enhance adhesion of the biocide to the seed, to provide slow-release of the biocide, and/or to provide color-coding for the treated seeds. Thus in an embodiment, a seed treatment composition comprises the biocide, the adjuvant, a polymeric binder, and water. The seed treatment composition can comprise, based on the total weight of the composition, 1 to 70 weight %, specifically 1 to 60 weight %, more specifically 5 to 50 weight % of the biocide; 1 to 90 weight %, specifically 10 to 60 weight %, more specifically 15 to 50 weight % of the adjuvant; 5 to 70 weight %, specifically 15 to 65 weight %, more specifically 15 to 60 weight % of the polymeric binder; and 1 to 90 weight %, specifically 10 to 60 weight %, more specifically 15 to 50 weight % water.

Examples of polymeric binders include polyesters, alkyds, polyamides, polycarbonates, polyureas, polyurethanes, (meth)acrylic polymers and copolymers, styrene copolymers, butadiene copolymers, polysaccharides such as starch and cellulose derivatives, vinyl alcohol, vinyl acetate and vinyl pyrrolidone polymers and copolymers, polyethers, epoxy resins, phenol-formaldehyde resins, melamine-formaldehyde resins, polyolefins, and copolymers and combinations of any of the foregoing. Specific polymers include styrene-(meth)acrylic polymers, styrene-butadiene polymers, vinyl acetate polymers, vinyl acetate-(meth)acrylic copolymers, ethylene-vinyl acetate copolymers, ethylene-vinyl acetate-vinyl chloride copolymers, styrene-maleic anhydride copolymers, cellulosic polymers such as ethyl cellulose, cellulose acetate, cellulose acetate butyrate (CAB), acetylated mono-, di-, and triglycerides, vinylpyrrolidone polymers and copolymers, vinyl acetate polymers and copolymers, poly(alkylene glycol) polymers such as poly(ethylene glycol), poly(propylene glycol), poly(ethylene glycol-propylene glycol), and poly(butylene glycol), poly(orthoesters), alkyd resins, and combinations thereof.

The polymeric binder can comprise a biodegradable polymer. A water-insoluble polymer is biodegradable if it decomposes over a period of several weeks when introduced into the environment. Examples of biodegradable polymers include biodegradable aliphatic polyesters, starch, polylactic acid, polylactic acid-starch blends, lactic acid-glycolic acid copolymers, polydioxanone, cellulose, cellulose derivatives such as ethyl cellulose, cellulose acetate butyrate (CAB), starch esters, starch ester-aliphatic polyester blends, modified corn starch, polycaprolactone, n-amyl methacrylate polymers, wood rosin, polyanhydrides, vinyl alcohol polymers, hydroxybutyrate polymers, hydroxybutyrate-valerate copolymers, and biodegradable aliphatic polyesters. Specifically, the polymeric binder comprises vinyl pyrrolidone polymers, vinyl acetate polymers and copolymers, vinyl alcohol polymers and copolymers, cellulose ethers, (meth)acrylic polymers and copolymers, and combinations thereof, more specifically (meth)acrylic copolymers.

The seed treatment compositions can be applied to plant propagation materials, particularly seeds, diluted or undiluted. The seed treatment compositions provide, after two-to-ten fold dilution, biocide concentrations of 0.01 to 60 weight %, specifically 0.1 to 40 weight %, in the diluted composition. Application of the seed treatment composition can be carried out before or during sowing. Methods for applying the seed treatment compositions onto plant propagation material, specifically seeds, include dressing, coating, pelleting, dusting, and soaking. The seed treatment composition can be applied to the plant propagation material so that germination is not induced, i.e. by seed dressing, pelleting, coating, and dusting.

The term “plant propagation material” as used herein denotes all the generative parts of a plant such as seeds and other vegetative plant material such as cuttings and tubers (e. g. potatoes), which can be used for the multiplication of the plant. This includes seeds, roots, fruits, tubers, bulbs, rhizomes, shoots, sprouts, and other parts of plants, including seedlings and young plants, which are to be transplanted after germination or after emergence from soil. These young plants can also be protected before transplantation by a total or partial treatment by immersion or pouring.

More specifically plant propagation material is the seed of various cultivated plants, for example cereals such as wheat, rye, barley, triticale, oats, rice; beet such as sugar beet or fodder beet; fruits such as pomes, stone fruits; soft fruits such as apples, pears, plums, peaches, almonds, cherries, strawberries, raspberries, blackberries or gooseberries; leguminous plants, such as lentils, peas, alfalfa or soybeans; oil plants, such as rape, oil seed rape/canola, mustard, olives, sunflowers, coconut, cocoa beans, castor oil plants, oil palms, ground nuts or soybeans; cucurbits, such as squashes, cucumber or melons, fiber plants, such as cotton, flax, hemp or jute, citrus fruit, such as oranges, lemons, grapefruits or mandarins; vegetables, such as spinach, lettuce, asparagus, cabbages, carrots, onions, tomatoes, potatoes, cucurbits or paprika; lauraceous plants, such as avocados, cinnamon or camphor, energy and raw material plants, such as corn, soybean, rape, sugar cane or oil palm; corn, tobacco, nuts, coffee, tea, bananas, vines (table grapes and juice grape vines), hops, turf, natural rubber plants; or ornamental and forestry plants such as flowers, shrubs, broad-leaved trees or evergreens such as conifers. Specifically the seed can be corn, sunflower, cereals such as wheat, rye, barley, triticale, oats, or rice, soybean, cotton, oil seed rape/canola, more specifically corn, sunflower, soybean, cereals such as wheat, rye, barley, triticale, oats, or rice, and most specifically corn, soybean, and cereals such as wheat, rye, barley, triticale, oats, or rice, still more specifically wheat, rye, barley, triticale, or oats.

The seed can also be from plants modified by breeding, mutagenesis, or genetic engineering, including commercial or developmental agricultural biotech products. Genetically modified plants are plants in which genetic material has been modified by the use of recombinant DNA techniques that under natural circumstances cannot readily be obtained by cross breeding, mutations, or natural recombination. For example, one or more genes have been integrated into the genetic material of a genetically modified plant in order to improve certain properties of the plant. Such genetic modifications also include but are not limited to targeted post-translational modification of protein(s), oligo- or polypeptides such as by glycosylation or polymer additions such as prenylated, acetylated, or farnesylated groups or polyethylene glycol groups.

The seeds can be from plants that have been rendered tolerant to specific classes of herbicides, that are by the use of recombinant DNA techniques capable of synthesizing one or more insecticidal proteins, specifically those known from the bacterial genus Bacillus, particularly from Bacillus thuringiensis, that are by the use of recombinant DNA techniques capable of synthesizing one or more proteins to increase the resistance or tolerance of those plants to bacterial, viral or fungal pathogens. The seeds can also be from plants that are by the use of recombinant DNA techniques capable of synthesizing one or more proteins to increase the productivity (e.g. biomass production, grain yield, starch content, oil content, or protein content), tolerance to drought, salinity, or other growth-limiting environmental factors, or tolerance to pests and fungal, bacterial or viral pathogens of those plants. The seeds can also be from plants that contain by the use of recombinant DNA techniques a modified amount of substances or new substances that improve human or animal nutrition, e.g. oil crops that produce health-promoting long-chain omega-3 fatty acids or unsaturated omega-9 fatty acids (e. g. NEXERA™ rape, DOW AgroSciences, Canada). The seeds can also be from plants that contain by the use of recombinant DNA techniques a modified amount of substances or new substances that improve the content of a desired raw material, e. g. potatoes that produce increased amounts of amylopectin (e. g. AMFLORA™ potato, BASF SE, Germany).

Biocide formulations of the invention can be in the form of any of the following: aqueous solutions, emulsifiable concentrates, emulsifiable granules, suspension concentrates, water dispersible granules, wettable powders, granules, oil in water emulsions, suspension emulsions, microemulsions, oil dispersions, and capsule suspensions.

### Examples

Solubility studies were conducted using three materials to determine their ability to dissolve various biocide active ingredients: Solvent I-formula 1(a); Solvent II-formula 1(b); and Solvent III-formula 3(a). Active ingredients were chosen from three classes of biocides and primarily reflected difficult to dissolve actives. The following biocides were evaluated.

| Herbicides | Insecticides | Fungicides |
|---|---|---|
| Glyphosate | Dimethoate | Flutriafol |
| 2,4-D | Gamma Cyhalothrin | Metalaxyl |
| Dicamba | | |
| Mesotrione | | |
| Halosulfuron | | |
| Fomesafen | | |

The approximate solubility of each technical grade active ingredient was determined in each of the solvents.

Experimental:

All examples were conducted on a 100 gram or 50 gram scale. The Dimethoate, 2,4-D, mesotrione and halosulfuron examples were conducted at 50 gram scale. All other examples were conducted on a 100 gram scale.

An amount of solvent was weighed into a 4 ounce jar (or 2 oz). An amount of active ingredient was weighed and added to the solvent in the jar. The jar was capped tightly and placed on a New Brunswick Scientific model C1 platform shaker. The samples were shaken at the 75% setting for 1 hour. During the mixing and after 1 hour each sample was examined and completeness of solubility noted. Using an iterative approach, an approximate solubility for each active ingredient in each of the solvents was determined. Each sample was allowed to sit undisturbed for 1 hour after mixing to determine if any crystallization would occur in those samples which dissolved.

Results:

The observed approximate solubility ranges of each active ingredient in the three solvents is shown below in Table 1. An indication of <2.5% means that the active did not dissolve at the minimum concentration tested of 2.5% solids by weight:

TABLE 1

| Active Ingredient | Solvent I | Solvent II | Solvent III |
|---|---|---|---|
| Glyphosate | <2.5% | <2.5% | <2.5% |
| 2,4-D | >2.5%; <10% | >10%; <20% | >20%; <40% |
| Dicamba | <10% | >30%; <40% | >40%; <50% |
| Mesotrione | <2.5% | <2.5% | >5%; <10% |
| Halosulfuron | <2.5% | <2.5% | >2.5%; <5% |
| Fomesafen | <2.5% | >2.5%; <10% | >5%; <10% |
| Dimethoate | >5%; <20% | >2%; <5% | >2%; <5% |
| Gamma cyhalothrin | >80% | >80% | >80% |
| Flutrifol | >2.5%; <5% | >2.5%; <5% | >5%; <20% |
| Metalaxyl | >20%; <30% | >20%; <30% | >40%; <50% |

In all but one active ingredient, Solvent III showed the best solvating properties of the three solvents for those active ingredients which were soluble.

The active ingredients that showed the greatest solubility were Gamma cyhalothrin, dicamba, and metalaxyl. Metalaxyl is used primarily for seed coatings so it's solubility in the more viscous Solvent I can be used for example, in a seed coating formulation.

Glyphosate, the active ingredient in Roundup herbicide was not very soluble in any of the solvents. Glyphosate, an aminophosphonic acid, is not soluble to any great extent in most solvents, including water. Typically, it is formulated as a salt dissolved in water with surfactants to facilitate penetration into the plant. While none of the solvents dissolved glyphosate acid, they did dissolve dicamba and 2,4-D which can be mixed with glyphosate to address glyphosate weed resistance.

The solubility of mesotrione in Solvent III was surprising. Mesotrione is not soluble in many solvents but has limited solubility in acetonitrile and acetone neither of which are suitable for agrochemical formulations. The observation that mesotrione is soluble in Solvent III is significant as this active ingredient is very difficult to formulate and difficult to suspend into water. Thus various formulations are possible with these solvents for this active ingredient which has favorable environmental and toxicological properties.

The terminology used herein is for the purpose of describing particular embodiments only and is not intended to be limiting. As used herein, the singular forms “a,” “an,” and “the” are intended to include the plural forms as well, unless the context clearly indicates otherwise. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The endpoints of all ranges directed to the same component or property are inclusive of the endpoint and independently combinable.

Unless otherwise defined, all terms (including technical and scientific terms) used herein have the same meaning as commonly understood by one of ordinary skill in the art to which this invention belongs. The compounds made by the above-described methods have, in embodiments, one or more isomers. Where an isomer can exist, it should be understood that the invention embodies methods that form any isomer thereof, including any stereoisomer, any conformational isomer, and any cis, trans isomer; isolated isomers thereof; and mixtures thereof.

Compounds are described using standard nomenclature. For example, any position not substituted by any indicated group is understood to have its valency filled by a bond as indicated, or a hydrogen atom. A dash (“−”) that is not between two letters or symbols is used to indicate a point of attachment for a substituent. For example, —CHO is attached through carbon of the carbonyl group. Alkyl groups can be straight-chained or branched. Throughout the specification, reference is made to various divalent groups. Such groups are the same as the monovalent groups that are similarly named, except an additional open valence replaces a hydrogen atom in the corresponding monovalent group. Divalent groups are indicated with an “-ene” suffix.

All cited patents, patent applications, and other references are incorporated herein by reference in their entirety.

The various embodiments described above are provided by way of illustration only and should not be construed to limit the claims attached hereto. The present compositions can comprise, consist of, or consist essentially of, any of the disclosed or recited elements. Thus, the invention illustratively disclosed herein can be suitably practiced in the absence of any element that is not specifically disclosed herein. Various modifications and changes will be recognized that can be made without following the example embodiments and applications illustrated and described herein, and without departing from the true spirit and scope of the following claims.

1. A biocide composition comprising:
a biocide, and
an adjuvant comprising a ketal having the formula (1) 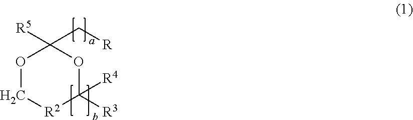
wherein
a is 0 or an integer of 1 to 12, specifically 1 to 6, more specifically 1 to 4, still more specifically 2 to 4;
b is 0, 1, or 2;
R is —C(═O)OR¹ or —CH₂OH;
R¹ is a C₁₋₁₈ alkyl or C₅₋₈ cycloaliphatic or heterocyclic group;
R² is a divalent C₁₋₈ alkylene group optionally substituted with up to 5 hydroxyl groups;
R³ and R⁴ are each independently hydrogen or C₁₋₆ alkyl; and
R⁵ is hydrogen or C₁₋₃ alkyl.

2. (canceled)

3. (canceled)

4. The biocide composition of claim 1, wherein R² is >CH—CH₃, >CH—CH₂OH, >C(CH₃)CH₂OH, >C(C₂H₅)CH₂OH, >C(CH₂OH)₂, >CH—CH(OH)—CH₂OH, or >CH—(CHOH)₃—CH₂OH.

5. (canceled)

6. (canceled)

7. (canceled)

8. The biocide composition of claim 1, wherein the ketal has the formula (1a) 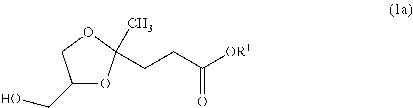
wherein R¹ is methyl, ethyl, n-propyl, n-butyl, 2-ethylhexyl, 1-nonyl, 3,5,5-trimethylhexyl, or tetrahydrofurfuryl,
or formula (1b) 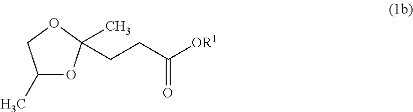
wherein R¹ is methyl, ethyl, n-propyl, n-butyl, 2-ethylhexyl, 1-nonyl, 3,5,5-trimethylhexyl, or tetrahydrofurfuryl,
or formula (1c) 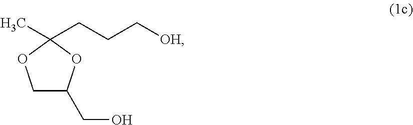
or formula (1d)

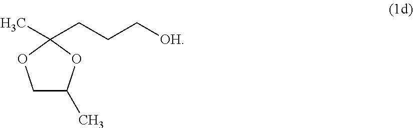

9. (canceled)

10. (canceled)

11. (canceled)

12. The biocide composition of claim 1, wherein the biocide comprises a herbicide, an insecticide, a fungicide, an algicide, a moluscicide, a miticide, a rodenticide, an antimicrobial, a plant growth regulator, or a combination thereof.

13. (canceled)

14. (canceled)

15. (canceled)

16. (canceled)

17. (canceled)

18. (canceled)

19. The biocide composition of claim 1, further comprising an oil, a solvent, a surfactant, a penetrant, water, a wetting agent, an anti-freeze, a preservative, stabilizing agent, a buffer, a rheology modifier, an anti-foam agent, a crystallization inhibitor, a fertilizer, or a combination comprising at least one of the foregoing.

20. (canceled)

21. (canceled)

22. (canceled)

23. (canceled)

24. (canceled)

25. (canceled)

26. (canceled)

27. (canceled)

28. (canceled)

29. (canceled)

30. The biocide composition of claim 1, wherein the composition is an emulsifiable concentrate, a suspension concentrate, a suspo-emulsion concentrate, a tank mix composition, or a ready-to-use liquid composition.

31. An emulsifiable concentrate comprising:
the biocide composition of claim 1;
a surfactant; and
optionally an oil,
wherein the emulsifiable concentrate comprises, based on the total weight of the composition:
0.01 to 99 weight %, specifically 0.05 to 75 weight %, more specifically 0.1 to 40 weight % of the biocide;
0.1 to 99 weight %, specifically 3 to 80 weight %, more specifically 10 to 70 weight % of the adjuvant;
0.1 to 15 weight %, specifically 1 to 10 weight %, more specifically 1 to 5 weight % of the surfactant; and
0 to 50 weight %, specifically 5 to 30 weight %, more specifically 10 to 25 weight % of the oil.

32. (canceled)

33. A suspension concentrate comprising:
the biocide composition of claim 1;
water;
a surfactant; and
optionally an oil,
wherein the suspension concentrate comprises, based on the total weight of the composition:
0.1 to 90 weight %, specifically 1 to 60 weight %, more specifically 10 to 50 weight % of the biocide;
5 to 60 weight %, specifically 10 to 50 weight %, more specifically 20 to 40 weight % of the adjuvant;
20 to 60 weight %, specifically 20 to 60 weight %, more specifically 20 to 50 weight % of water;
1 to 35 weight %, specifically 2 to 20 weight %, more specifically 3 to 12 weight % of the surfactant; and
0 to 70 weight %, specifically 10 to 60 weight %, more specifically 20 to 50 weight % of the oil.

34. (canceled)

35. A tank mix composition for foliar or soil application, comprising:
the biocide composition of claim 1;
water;
a surfactant; and
optionally an oil,
wherein the tank mix composition comprises, based on the total weight of the composition:
0.0005 to 20 weight %, specifically 0.002 to 10 weight %, more specifically 0.005 to 2 weight % of the biocide;
0.0005 to 80 weight %, specifically 0.002 to 40 weight %, more specifically 0.005 to 8 weight % of the adjuvant;
40 to 99 weight %, specifically 50 to 90 weight %, more specifically 50 to 80 weight % water;
0.1 to 30 weight %, specifically 1 to 25 weight %, more specifically 5 to 20 weight % of the surfactant; and
0 to 20 weight %, specifically 1 to 15 weight %, more specifically 1 to 10 weight % of the oil.

36. (canceled)

37. A ready-to-use liquid composition comprising:
the biocide composition of claim 1;
a solvent other than the adjuvant; and
optionally a thickening agent, a propellant, an attractant, or a combination thereof;
wherein the solvent is 90% vaporized within 5 minutes of application of the composition to a surface,
wherein the ready-to-use liquid comprises, based on the total weight of the composition:
0.0005 to 2 weight %, specifically 0.002 to 1 weight %, more specifically 0.005 to 0.5 weight % of the biocide;
10 to 60 weight %, specifically 20 to 50 weight %, more specifically 30 to 40 weight % of the adjuvant;
10 to 60 weight %, specifically 20 to 50 weight %, more specifically 30 to 40 weight % of the solvent;
0 to 5 weight %, specifically 0.1 to 1 weight %, more specifically 0.5 to 1 weight % of the thickening agent;
0 to 75 weight %, specifically 5 to 50 weight %, more specifically 10 to 30 weight % of the propellant; and
0 to 70 weight %, specifically 5 to 60 weight %, more specifically 10 to 40 weight % of the attractant.

38. (canceled)

39. A composition, comprising an adjuvant comprising a ketal having the formula (1) 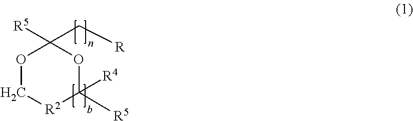
wherein
a is 0 or an integer of 1 to 12, specifically 1 to 6, more specifically 1 to 4, still more specifically 2 to 4;
b is 0, 1, or 2;
R is —C(═O)OR¹ or —CH₂OH;
R¹ is a C₁₋₁₈ alkyl or C₅₋₈ cycloaliphatic or heterocyclic group;
R² is a divalent C₁₋₈ alkylene group optionally substituted with up to 5 hydroxyl groups;
R³ and R⁴ are each independently hydrogen or C₁₋₆ alkyl; and
R⁵ is hydrogen or C₁₋₃ alkyl;
a compound having the formula (6) 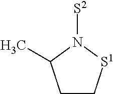
wherein S¹ is >C═O (carbonyl) or >CH₂ (methylene), and S² is C₁₋₁₂ alkyl, C₆₋₂₀ cycloalkyl, or C₆₋₂₀ aryl;
a compound having the formula (7) or (8) 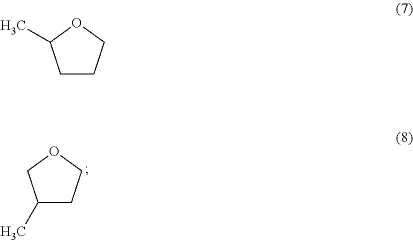
a compound having the formula (11) 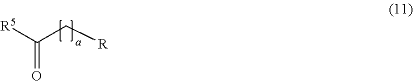
wherein
a is 0 or an integer of 1 to 12;
R is —CH₂OH; and
R⁵ is hydrogen or C₁₋₃ alkyl;
a compound having the formula (3) 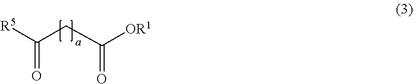
wherein
a is 0 or an integer of 1 to 12;
R¹ is a C₁₋₁₈ alkyl or C₅₋₈ cycloaliphatic or heterocyclic group; and
R⁵ is hydrogen or C₁₋₃ alkyl;
or a combination thereof,
wherein the composition is a crop oil concentrate or a seed treatment composition.

40. (canceled)

41. (canceled)

42. The composition of claim 39 comprising, based on the total weight of the composition:
1 to 99 weight %, specifically 10 to 90 weight %, more specifically 20 to 80 weight % of the adjuvant;
1 to 40 weight %, specifically 2 to 20 weight %, more specifically 2 to 10 weight % of a surfactant; and
0 to 95 weight %, specifically 5 to 90 weight %, more specifically 10 to 80 weight % of an oil,
wherein the composition is a crop oil concentrate.

43. (canceled)

44. (canceled)

45. (canceled)

46. The seed treatment composition of claim 39, further comprising a biocide, a polymeric binder, water or a combination thereof and comprising, based on the total weight of the seed treatment composition:
1 to 70 weight %, specifically 1 to 60 weight %, more specifically 5 to 50 weight % of the biocide;
1 to 90 weight %, specifically 10 to 60 weight %, more specifically 15 to 50 weight % of the adjuvant;
5 to 70 weight %, specifically 15 to 65 weight %, more specifically 15 to 60 weight % of the polymeric binder; and
1 to 90 weight %, specifically 10 to 60 weight %, more specifically 15 to 50 weight % water.

47. A method of controlling a pest, the method comprising contacting the pest or the locus of the pest with the biocide composition of claim 1, in an amount effective to control the pest.

48. (canceled)

49. (canceled)

50. (canceled)

51. A biocide composition comprising:
a biocide, and
an adjuvant comprising a compound having the formula (11) 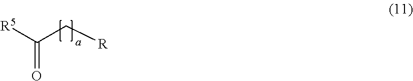
wherein
a is 0 or an integer of 1 to 12;
R is —CH₂OH; and
R⁵ is hydrogen or C₁₋₃ alkyl,
a compound having the formula (3) 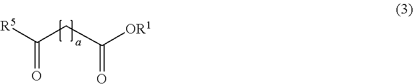
wherein
a is 0or an integer of 1 to 12;
R¹ is a C₁₋₁₈ alkyl or C₅₋₈ cycloaliphatic or heterocyclic group; and
R⁵ is hydrogen or C₁₋₃ alkyl,
a compound having the formula (6) 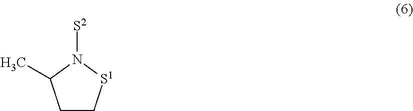
wherein S¹ is >C═O (carbonyl) or >CH₂ (methylene), and S² is C₁₋₁₂ alkyl, C₆₋₂₀ cycloalkyl, or C₆₋₂₀ aryl;
a compound having the formula (7) 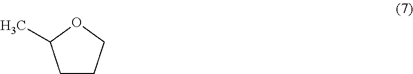
a compound having the formula (8) 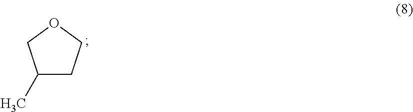
or a combination thereof.

52. (canceled)

53. The biocide composition of claim 51, wherein R⁵ in formula (11) is methyl; and a in formula (11) is 2 or 3.

54. (canceled)

55. (canceled)

56. (canceled)

57. The biocide composition of claim 51, wherein R⁵ in formula (3) is methyl; and a in formula (3) is 1, 2, 3, or 4.

58. (canceled)

59. (canceled)

60. (canceled)

61. (canceled)

62. (canceled)

63. (canceled)

64. (canceled)

65. (canceled)

66. (canceled)

67. The biocide composition of claim 51, wherein the adjuvant comprises the compound having the formula (6) wherein S² is methyl, ethyl, butyl, octyl, decyl, dodecyl, phenyl, 4-methylphenyl, or cyclohexyl.

68. The biocide composition of claim 51, wherein the adjuvant comprises the compound having the formula (6) wherein S¹ is >C═O (carbonyl) or >CH₂ (methylene) and S² is methyl, ethyl, butyl, octyl, decyl, dodecyl, phenyl, 4-methylphenyl, or cyclohexyl.

69. (canceled)

70. The biocide composition of claim 51, wherein the adjuvant comprises the compound of formula (9) 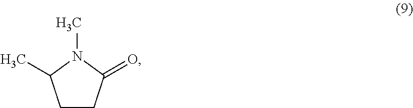
or compound of formula (10)

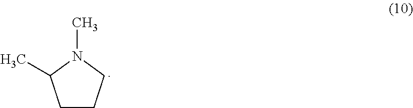

71. (canceled)

72. (canceled)

73. (canceled)
